(19) United States
(12) Patent Application Publication
(10) Pub. No.: US 2023/0118974 A1
(43) Pub. Date: Apr. 20, 2023

(54) TECHNOLOGIES FOR GENETIC ENGINEERING DETECTION

(71) Applicant: BATTELLE MEMORIAL INSTITUTE, Columbus, OH (US)

(72) Inventors: Omar P. TABBAA, Columbus, OH (US); Craig M. BARTLING, Columbus, OH (US); Brett R. FOWLE, Columbus, OH (US); Patrick FULLERTON, Columbus, OH (US); Bryan GEMLER, Columbus, OH (US); Carrie HOWLAND, Columbus, OH (US); Danielle J. HUK, Columbus, OH (US); Zachary R. SHANK, Columbus, OH (US)

(21) Appl. No.: 18/047,818

(22) Filed: Oct. 19, 2022

Related U.S. Application Data
(60) Provisional application No. 63/257,500, filed on Oct. 19, 2021.

(51) Int. Cl. G16B 30/10

(52) U.S. Cl. CPC G16B 30/10

(57) ABSTRACT

Technologies for identifying genetic engineering proteins, organisms, and context signatures include a computing device that may be in communication with multiple client devices. The technologies include receiving a query sequence for a biological specimen, determining an alignment of the query sequence for regions of interest, and determining whether a match exists upstream or downstream from the region of interest in a predetermined database of genetic engineering context signatures. The search range may be predetermined based on each context signature. The technologies further include determining an alignment of the query sequence against a predetermined database of sequences indicative of genetic engineering, and determining whether a similarity score of the alignment exceeds a predetermined threshold. The database may include a genetic engineering protein database or a genetic engineering organism database. Other embodiments are described and claimed.

### CROSS-REFERENCE TO RELATED APPLICATION

This application claims the benefit of and priority to U.S. Patent Application No. 63/257,500, entitled “Genetic Engineering Detection Module,” which was filed on Oct. 19, 2021, which is incorporated herein by reference in its entirety.

### BACKGROUND

Recent estimates suggest that there are roughly one trillion distinct, naturally occurring microbial species present in the environment, of which only a fraction of a percent have been cultured in order to be studied in greater detail in a laboratory setting. Known human pathogens comprise an even smaller percentage of this figure, where there are roughly 1,400 different pathogenic species identified, to date, using presently available bioinformatics and molecular characterization methods. From both an epidemiological and ecological perspective, detecting and tracking naturally occurring human pathogens can be quite cumbersome given the sheer volume and diversity of environmentally derived microorganisms.

Advances in genetic engineering have made it possible to rapidly and efficiently impart novel cellular functionality into environmentally derived host cells. These breakthroughs have provided considerable benefit to multiple facets of human life, such as creating more efficient processes for drug discovery and production, as well as increasing crop yields under poor growing conditions. However, from the perspective of biosecurity, there is the potential for the use of genetic engineering to create biological weapons, and typical methods to differentiate between environmentally derived pathogens and microbes that have been intentionally modified are limited. This has the capacity to create an unpleasant public health scenario in the event of a deliberate engineered pathogen release due to the likelihood for bypassing detection via traditional screening methods. Further, methods for molecular forensic investigations into intentional pathogen creation are still emerging.

### SUMMARY

According to one aspect of the disclosure, a computing device for identifying genetic engineering comprises a query mapper and a genetic engineering context module. The query mapper is to receive a query sequence for a biological specimen and determine an alignment of the query sequence for regions of interest, wherein each region of interest comprises a part of a whole protein translated region. The genetic engineering context module is to determine whether a match for a genetic engineering context signature exists adjacent to a region of interest of the query sequence, wherein the genetic engineering context signature comprises a sequence selected from a predetermined database of sequences indicative of genetic engineering context signatures, and indicate presence of the genetic engineering context signature in response to a determination that the match exists. In an embodiment, the query sequence comprises an amino acid sequence or a nucleotide sequence.

In an embodiment, to determine whether the match for the genetic engineering context signature exists adjacent to the region of interest comprises to search upstream or downstream of the region of interest in the query sequence. In an embodiment, to search upstream or downstream of the region of interest comprises to search over a predetermined search range, wherein the predetermined search range is associated with the genetic engineering context signature.

In an embodiment, the region of interest comprises a protein that is indicative of genetic engineering. In an embodiment, the region of interest comprises a predetermined protein sequence of interest. In an embodiment, the region of interest comprises a protein associated with a biologically threatening function. In an embodiment the region of interest comprises a predetermined protein.

In an embodiment, the genetic engineering context signature comprises an upstream regulatory element, a downstream regulatory element, or a tag. In an embodiment, the genetic engineering context signature comprises an upstream regulatory element, wherein the upstream regulatory element comprises a promoter, a ribosome binding site, an operator that contributes to transcript regulation, or an enhancer. In an embodiment, the genetic engineering context signature comprises a downstream regulatory element, wherein the downstream regulatory element comprises a terminator, a polyA site, a woodchuck hepatitis virus posttranscriptional regulatory element (WPRE), a CTE, or an LTR. In an embodiment, the genetic engineering context signature comprises a tag, wherein the tag comprises a purification/epitope tag, a cleavage sequence, or a targeting sequence.

According to another aspect, a computing device for identifying genetic engineering comprises a query mapper and a genetic engineering detection module. The query mapper is to receive a query sequence for a biological specimen and determine an alignment of the query sequence against a predetermined database of sequences indicative of genetic engineering. The genetic engineering detection module is to determine whether a similarity score associated with the alignment has a predetermined relationship to a predetermined threshold, and indicate presence of genetic engineering in response to a determination that the similarity score has the predetermined relationship to the predetermined threshold. In an embodiment, the query sequence comprises an amino acid sequence or a nucleotide sequence.

In an embodiment, the predetermined database of sequences indicative of genetic engineering comprises a database indicative of proteins, wherein each protein of the database is indicative of genetic engineering. In an embodiment, each protein comprises a selectable marker, a reporter, a transcription regulator, a post-translation regulator, a gene editing/delivery protein, a plasmid replication protein, a protein coupler, a protein folder, a polymerase, or a viral packaging/assembly protein. In an embodiment, the protein comprises a selectable marker, and wherein the selectable marker comprises a gene-encoded function that confers a selectable trait, wherein the trait comprises a specific antibiotic resistance, a toxin, an antitoxin, or an auxotrophy marker. In an embodiment, the protein comprises a reporter, and wherein the reporter comprises an enzymatic reporter, a direct optical reporter, or an analyte sensor. In an embodiment, the protein comprises a transcription regulator, wherein the transcription regulator comprises a repressor or an activator.

In an embodiment, the predetermined database of sequences indicative of genetic engineering comprises a database indicative of organisms, wherein each organism of the database is indicative of genetic engineering. In an embodiment, the organism comprises a model organism, a delivery organism, a chassis/cloning organism, or a targeted protein overexpression organism.

According to another aspect, a method for identifying genetic engineering comprises receiving, by a computing device, a query sequence for a biological specimen; determining, by the computing device, an alignment of the query sequence for regions of interest, wherein each region of interest comprises a part of a whole protein translated region; determining, by the computing device, whether a match for a genetic engineering context signature exists adjacent to a region of interest of the query sequence, wherein the genetic engineering context signature comprises a sequence selected from a predetermined database of sequences indicative of genetic engineering context signatures; and indicating, by the computing device, presence of the genetic engineering context signature in response to determining that the match exists. In an embodiment, the query sequence comprises an amino acid sequence or a nucleotide sequence.

In an embodiment, determining whether the match for the genetic engineering context signature exists adjacent to the region of interest comprises searching upstream or downstream of the region of interest in the query sequence. In an embodiment, searching upstream or downstream of the region of interest comprises searching over a predetermined search range, wherein the predetermined search range is associated with the genetic engineering context signature.

In an embodiment, the region of interest comprises a protein that is indicative of genetic engineering. In an embodiment, the region of interest comprises a predetermined protein sequence of interest. In an embodiment, the region of interest comprises a protein associated with a biologically threatening function. In an embodiment, the region of interest comprises a predetermined protein.

In an embodiment, the genetic engineering context signature comprises an upstream regulatory element, a downstream regulatory element, or a tag. In an embodiment, the genetic engineering context signature comprises an upstream regulatory element, wherein the upstream regulatory element comprises a promoter, a ribosome binding site, an operator that contributes to transcript regulation, or an enhancer. In an embodiment, the genetic engineering context signature comprises a downstream regulatory element, wherein the downstream regulatory element comprises a terminator, a polyA site, a woodchuck hepatitis virus posttranscriptional regulatory element (WPRE), a CTE, or an LTR. In an embodiment, the genetic engineering context signature comprises a tag, wherein the tag comprises a purification/epitope tag, a cleavage sequence, or a targeting sequence.

According to another aspect, a method for identifying genetic engineering comprises receiving, by a computing device, a query sequence for a biological specimen; determining, by the computing device, an alignment of the query sequence against a predetermined database of sequences indicative of genetic engineering; determining, by the computing device, whether a similarity score associated with the alignment has a predetermined relationship to a predetermined threshold; and indicating, by the computing device, presence of genetic engineering in response to determining that the similarity score has the predetermined relationship to the predetermined threshold. In an embodiment, the query sequence comprises an amino acid sequence or a nucleotide sequence.

In an embodiment, the predetermined database of sequences indicative of genetic engineering comprises a database indicative of proteins, wherein each protein of the database is indicative of genetic engineering. In an embodiment, each protein comprises a selectable marker, a reporter, a transcription regulator, a post-translation regulator, a gene editing/delivery protein, a plasmid replication protein, a protein coupler, a protein folder, a polymerase, or a viral packaging/assembly protein. In an embodiment, the protein comprises a selectable marker, and wherein the selectable marker comprises a gene-encoded function that confers a selectable trait, wherein the trait comprises a specific antibiotic resistance, a toxin, an antitoxin, or an auxotrophy marker. In an embodiment, the protein comprises a reporter, and wherein the reporter comprises an enzymatic reporter, a direct optical reporter, or an analyte sensor. In an embodiment, the protein comprises a transcription regulator, wherein the transcription regulator comprises a repressor or an activator.

In an embodiment, the predetermined database of sequences indicative of genetic engineering comprises a database indicative of organisms, wherein each organism of the database is indicative of genetic engineering. In an embodiment, the organism comprises a model organism, a delivery organism, a chassis/cloning organism, or a targeted protein overexpression organism.

### BRIEF DESCRIPTION OF THE DRAWINGS

The detailed description particularly refers to the accompanying figures in which:

FIG. 1 is a simplified block diagram of at least one embodiment of a system for detecting genetic engineering proteins, organisms, and context signatures;

FIG. 2 is a simplified block diagram of at least one embodiment of an environment that may be established by a computing device of the system of FIG. 1;

FIGS. 3 and 4 are a simplified flow diagram of at least one embodiment of a method for detecting genetic engineering proteins, organism, and context signatures that may be executed by the computing device of FIGS. 1 and 2;

FIG. 5 is a schematic diagram illustrating upstream searching for nucleotide genetic engineering context signatures;

FIG. 6 is a schematic diagram illustrating downstream searching for nucleotide genetic engineering context signatures; and

FIG. 7 is a schematic diagram illustrating upstream and downstream searching for amino acid genetic engineering context signatures.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but to the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); or (A, B, and C). Similarly, items listed in the form of “at least one of A, B, or C” can mean (A); (B); (C): (A and B); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

The technology described herein may be used for taxonomic identification and/or for identification of genetically engineered plant, animal, or human pathogens, for example. In one embodiment, the technology described herein may comprise identifying a query sequence wherein the query sequence may comprise a nucleic acid sequence or a protein coding sequence (i.e., an amino acid sequence) from a pathogenic organism selected, for example, from the group consisting of bacteria, archea, fungi, eukaryotes, and viruses. In various embodiments, the query sequence can comprise a sequence from a genetic engineering context protein, a genetic engineering organism, and/or a genetic engineering context signature. The identification of the plant, animal, or human pathogen as being genetically engineered involves comparison of the query sequence from a specimen from a plant, animal, or human, or from the environment against one or more predetermined databases of known genetic engineering proteins, genetic engineering organisms, and/or genetic engineering context signatures to identify the plant, animal, or human pathogen as being a genetically engineered pathogen. Accordingly, the technology allows differentiation between engineered and non-engineered organisms, including pathogens, through nucleotide and/or amino acid sequence comparisons. The technology used for this comparison is described in more detail below.

In various embodiments, a biological or environmental specimen can be tested for the presence of a genetic engineering context protein, a genetic engineering organism, and/or a genetic engineering context signature using the technology described herein. The biological specimen can comprise human or animal body fluids including, but not limited to, urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, a stool sample, reproductive tract secretions, such as seminal fluid, lymph fluid, and whole blood, serum, or plasma, or any other suitable human or animal biological specimen. In additional embodiments, human or animal tissue samples that can be tested can include tissue biopsies of hospital patients or out-patients and autopsy specimens, or an animal tissue specimen. As used herein, the term “tissue” includes, but is not limited to, biopsies, autopsy specimens, cell extracts, hair, tissue sections, aspirates, tissue swabs, and fine needle aspirates.

In various illustrative embodiments, the biological specimen can be a plant sample from any part of a plant such as the stem, a leaf, a flower, a bud, a calyx, a corolla, the roots, a fruit, etc. In another embodiment, the specimen can be an environmental specimen selected from the group consisting of a soil sample, a water sample, a food sample, an air sample, an industrial waste sample, an agricultural sample, a surface wipe sample, a dust sample, a hair sample, or any other suitable environmental specimen.

In one illustrative aspect, the nucleic acids and/or proteins in the specimen are extracted and purified for analysis of a query sequence. In various embodiments, the preparation of the nucleic acids (e.g., DNA or RNA) can involve rupturing the cells that contain the nucleic acids and isolating and purifying the nucleic acids (e.g., DNA or RNA) from the lysate. Techniques for rupturing cells and for isolation and purification of nucleic acids (e.g., DNA or RNA) are well-known in the art. In one embodiment, for example, nucleic acids may be isolated and purified by rupturing cells using a detergent or a solvent, such as phenol-chloroform. In another aspect, nucleic acids (e.g., DNA or RNA) may be separated from the lysate by physical methods including, but not limited to, centrifugation, pressure techniques, or by using a substance with an affinity for nucleic acids (e.g., DNA or RNA), such as, for example, beads that bind nucleic acids. In one embodiment, after sufficient washing, the isolated, purified nucleic acids may be suspended in either water or a buffer. In one embodiment, “isolated” means that the nucleic acids or proteins are removed from their normal environment (e.g., a nucleic acid is removed from the genome of an organism). In another aspect, “purified” means the nucleic acids or proteins are substantially free of other cellular material, or culture medium, or other chemicals used in the extraction process. In other embodiments, commercial kits are available, such as Qiagen™ (e.g., Qiagen DNeasy PowerSoil Kit™), Nuclisensm™, and Wizard™ (Promega), and Promegam™ for extraction and purification of nucleic acids. In yet another embodiment, a protein can be purified and sequenced or the amino acid sequence of a protein can be derived from a nucleic acid sequence. Methods for preparing nucleic acids and for purifying and sequencing proteins are also described in Green and Sambrook, “Molecular Cloning: A Laboratory Manual”, 4th Edition, Cold Spring Harbor Laboratory Press, (2012), incorporated herein by reference.

In one illustrative aspect, the query sequence can be identified after sequencing the nucleic acids by using any suitable sequencing method including Next Generation Sequencing (e.g., using Illumina, ThermoFisher, or PacBio or Oxford Nanopore Technologies sequencing platforms), sequencing by synthesis, pyrosequencing, nanopore sequencing, or modifications or combinations thereof can be used. Methods for sequencing nucleic acids and proteins are also well-known in the art and are described in Sambrook et al., “Molecular Cloning: A Laboratory Manual”, Cold Spring Harbor Laboratory Press, incorporated herein by reference.

Exemplary genetically engineered pathogens from which a query sequence may be obtained include, but are not limited to, genetically engineered fungi such fungi selected from the group consisting of Absidia coerulea, Absidia glauca, Absidia corymbifera, Acremonium strictum, Alternaria alternata, Apophysomyces elegans, Saksena vasiformis, Aspergillus flavus, Aspergillus oryzae, Aspergillus fumigatus, Neosartoryta fischeri, Aspergillus niger, Aspergillus foetidus, Aspergillus phoenicus, Aspergillus nomius, Aspergillus ochraceus, Aspergillus ostianus, Aspergillus auricomus, Aspergillus parasiticus, Aspergillus sojae, Aspergillus restrictus, Aspergillus caesillus, Aspergillus conicus, Aspergillus sydowii, Aspergillus tamarii, Aspergillus terreus, Aspergillus ustus, Aspergillus versicolor, Aspergillus ustus, Aspergillus versicolor, Chaetomium globosum, Cladosporium cladosporioides, Cladosporium herbarum, Cladosporium sphaerospermum, Conidiobolus coronatus, Conidiobolus incongruus, Cunninghamella elegans, Emericella nidulans, Emericella rugulosa, Emericilla quadrilineata, Apicoccum nigrum, Eurotium amstelodami, Eurotium chevalieri, Eurotium herbariorum, Eurotium rubrum, Eurotium repens, Geotrichum candidum, Geotrichum klebahnii, Memnoniella echinata, Mortierella polycephala, Mortierella wolfii, Mucor mucedo, Mucor amphibiorum, Mucor circinelloides, Mucor heimalis, Mucor indicus, Mucor racemosus, Mucor ramosissimus, Rhizopus azygosporous, Rhizopus homothalicus, Rhizopus micro sporus , Rhizopus oligosporus, Rhizopus oryzae, Myrothecium verruc aria, Myrothecium roridum, Paecilomyces lilacinus, Paecilomyces variotii, Penicillium freii, Penicillium verrucosum, Penicillium hirsutum, Penicillium alberechii, Penicillum aurantiogriseum, Penicillium polonicum, Penicillium viridicatum, Penicillium hirsutum, Penicillium brevicompactum, Penicillium chrysogenum, Penicillium griseofulvum, Penicillium glandicola, Penicillium coprophilum, Eupenicillium crustaceum, Eupenicillium egyptiacum, Penicillium crustosum, Penicillium citrinum, Penicillium sartoryi, Penicillium westlingi, Penicillium corylophilum, Penicillium decumbens, Penicillium echinulatum, Penicillium solitum, Penicillium camembertii, Penicillium commune, Penicillium echinulatum, Penicillium sclerotigenum, Penicillium italicum, Penicillium expansum, Penicillium fellutanum, Penicillium charlesii, Penicillium janthinellum, Penicillium raperi, Penicillium madriti, Penicillium gladioli, Penicillium oxalicum, Penicillium roquefortii, Penicillium simplicissimum, Penicillium ochrochloron, Penicillium spinulosum, Penicillium glabrum, Penicillum thomii, Penicillium pupurescens, Eupenicillium lapidosum, Rhizomucor miehei, Rhizomucor pusillus, Rhizomucor variabilis, Rhizopus stolonifer, Scopulariopsis asperula, Scopulariopsis brevicaulis, Scopulariopsis fusca, Scopulariopsis brumptii, Scopulariopsis chartarum, Scopulariopsis sphaerospora, Trichoderma asperellum, Trichoderma hamatum, Trichoderma viride, Trichoderma harzianum, Trichoderma longibrachiatum, Trichoderma citroviride, Trichoderma atroviride, Trichoderma koningii, Ulocladium atrum, Ulocladium chartarum, Ulocladium botrytis, Wallemia sebi, Stachybotrys chartarum, and the like.

Exemplary genetically engineered bacterial pathogens can be selected from Gram-negative and Gram-positive cocci and bacilli, acid-fast bacteria, and can comprise antibiotic-resistant bacteria, or any other genetically engineered bacterial pathogen. In another illustrative aspect, the genetically engineered bacteria can be selected from the group consisting of Pseudomonas species, Staphylococcus species, Streptococcus species, Escherichia species, Haemophilus species, Neisseria species, Chlamydia species, Helicobacter species, Campylobacter species, Salmonella species, Shigella species, Clostridium species, Treponema species, Ureaplasma species, Listeria species, Legionella species, Mycoplasma species, and Mycobacterium species, or the group consisting of S. aureus, P. aeruginosa, and E. coli.

In another aspect, the genetically engineered pathogen can be a virus and the virus can be selected from DNA and RNA viruses or can be selected from the group consisting of papilloma viruses, parvoviruses, adenoviruses, herpesviruses, vaccinia viruses, arenaviruses, coronaviruses, rhinoviruses, respiratory syncytial viruses, influenza viruses, picornaviruses, paramyxoviruses, reoviruses, retroviruses, and rhabdoviruses. In another illustrative embodiment, mixtures of any of these genetically engineered pathogens can be identified as being present in the specimen. In yet another embodiment, the specimen to be tested comprises eukaryotic cells.

In various embodiments described herein, to identify a query sequence from a genetically engineered plant, animal, or human pathogen, for example, a genetic engineering context protein, a genetic engineering organism, or a genetic engineering context signature can be identified. Genetic engineering context proteins are proteins indicative of genetic engineering, such as those used for selection, reporting, protein purification, etc. The coding sequences for these proteins have been documented in the literature as being a component of a vector and/or another module used during genetic engineering.

In illustrative embodiments, genetic engineering context proteins can be selected from a selectable marker (i.e., a gene-encoded function that confers a selectable trait) such as antibiotic resistance, toxin/antitoxin combinations (i.e., a selectable marker composed of a toxin gene and its cognate antitoxin), auxotrophy, such as a selectable marker that requires a specific metabolite for growth or death (e.g., uracil auxotroph systems that enable selection through metabolic manipulation of the cell), or a reporter which is a gene-encoded function that is used in genetic engineering to indicate target gene transformation, expression of a target gene, gene-gene interaction, or activity of a promoter or other genetic element. Reporter gene activities are easily measured through optical or other means, such as enzymatic assays where an enzymatic reporter is used (e.g., beta galactosidase). A reporter can be a direct optical reporter (e.g., a luminescent protein) or an analyte sensor (i.e., a type of reporter in which the encoded gene is a sensor of a specific analyte (e.g., calmodulin)). Exemplary detectable optical reporters include fluorescent dyes such as beta-glucuronidase (GUS) of the uid.A locus of E. coli, chloramphenicol acetyl transferase from Tn9 of E. coli, the green fluorescent protein (GFP) from the bioluminescent jellyfish Aequorea, and the luciferase genes from the firefly Photinus pyralis.

Other exemplary genetic engineering context proteins include, but are not limited to, transcription regulators for repression or activation of gene expression through binding of DNA elements upstream of the gene, repressors which are regulatory proteins that bind to an operator (genetic sequence between the promoter and the expressed genes in an operon) thereby impeding RNA polymerase and thus gene expression, activators which are regulatory proteins that increase gene transcription typically by binding to DNA elements upstream of a gene, and post-translational regulators, such as the ClpXP system or ubiquitin.

In the embodiment where the genetic engineering context protein is a selectable marker, the selectable marker can be an antibiotic resistance gene or a gene capable of complementing a metabolic deficiency, such as in tryptophan or histidine deficient mutants. Exemplary selectable markers can include URA3, LEU2, HIS3, TRP1, HIS4, ARG4, or antibiotic resistance markers, such as ampicillin resistance markers (e.g., AmpR), neomycin resistance markers (e.g., NeoR), G418, bleomycin resistance markers, hygromycin resistance markers, chloramphenicol resistance markers, methotrexate resistance markers, and kanamycin resistance markers.

In other embodiments, a genetic engineering context protein can comprise a gene editing/delivery system, such as nucleases and recombinases (e.g., CRISPR, TALENS, exonucleases, Cre recombinase, and histone H2B). In further embodiments, a genetic engineering context protein can comprise a plasmid replication protein, a protein coupler which leverages specific protein-protein or protein-ligand affinity interaction (e.g., streptavidin or maltose binding protein), a display protein (e.g., coat protein for phage display), a protein recombinantly produced for affinity resins (e.g., Protein A), a protein folder, a polymerase (e.g., a T7 polymerase), or a viral packaging/assembly protein.

In another embodiment, to identify a query sequence from a genetically engineered plant, animal, or human pathogen, for example, a genetic engineering context signature can be identified. A genetic engineering context signature can be a small nucleic acid or amino acid sequence found either upstream or downstream of one or more coding sequences that regulates transcription of the gene and/or aids in cellular localization or purification of the protein product. These sequences have been documented in the literature.

In various aspects, genetic engineering context signatures can include, but are not limited to, an upstream regulatory element that regulates transcription and/or protein expression, a promoter, a ribosome binding site, an operator that contributes to transcription regulation (e.g., the lac operator which binds to the lac repressor), TRE response elements, “LTR” features, 5′ UTRs, insulators, enhancers, downstream regulatory elements, terminators, a polyA site/polyA signal which can be important for nuclear export, translation, and stability of mRNA, and other downstream transcription regulatory elements (e.g., Woodchuck Hepatitis Virus Posttranscriptional Regulatory Element (WPRE) which enhances expression, 3′ UTRs, insulators, etc.). In other embodiments, genetic engineering context signatures can include a tag, such as an amino acid sequence found at the N-terminal or C-terminal end of an engineered protein to target the protein to specific cellular locations and/or to aid in protein purification (e.g., His×6 tag, HA tag, etc.). A genetic engineering context signature can also be a cleavage sequence (e.g., TEV protease or self-cleaving peptide), or a targeting sequence.

Other exemplary genetic engineering context signatures can include a localization signal (e.g., a nuclear localization signal, a mitochondrial localization signal, or a plastid localization signal), a transit or targeting peptide, a cell-penetrating peptide, an endosomal escape peptide, and a restriction enzyme cleavage site sequence.

In one embodiment, the genetic engineering context signature can be a promoter. Exemplary promoters may be selected from the group consisting of a of a pol III promoter, a pol II promoter, a pol I promoter, a U6 promoter, an H1 promoter, a Rous sarcoma virus (RSV) LTR promoter, a cytomegalovirus (CMV) promoter, an SV40 promoter, a dihydrofolate reductase promoter, a beta-actin promoter, a phosphoglycerol kinase (PGK) promoter, an AOX promoter, an EF1a promoter, a pol II promoter, a CaMV promoter, a maize chloroplast aldolase promoter, an opaline synthase (NOS) promoter, an octapine synthase (OCS) promoter, a figwort mosaic virus (FMV) promoter, a RUBISCO promoter, a pyruvate phosphate dikinase (PDK) promoter, a T7 promoter, a 26S promoter, a CsVMV promoter, a lac promoter, an AMP promoter, a mannopine synthase promoter, a maize ubiquitin promoter, an Arabidopsis ubiquitin promoter, a 35T promoter, and an AtUbi10 promoter.

In embodiments where the genetic engineering context signature is a termination signal sequence, the terminator can be a U6 poly-T terminator, an SV40 terminator, an hGH terminator, a BGH terminator, an rbGlob terminator, a synthetic terminator functional in a eukaryotic cell, or a 3′ element from an Agrobacterium sp. gene.

In another embodiment, the genetic engineering context signature is a sequence from an expression vector such as a viral vector selected from the group consisting of adenoviruses, lentiviruses, adeno-associated viruses, retroviruses, geminiviruses, begomoviruses, tobamoviruses, potex viruses, comoviruses, wheat streak mosaic virus, barley stripe mosaic virus, bean yellow dwarf virus, bean pod mottle virus, cabbage leaf curl virus, beet curly top virus, tobacco yellow dwarf virus, tobacco rattle virus, potato virus X, and cowpea mosaic virus. In other embodiments, the genetic engineering context signature can be a sequence from a bacterial vector selected from the group consisting of Agrobacterium sp., Rhizobium sp., Sinorhizobium (Ensifer) sp., Mesorhizobium sp., Bradyrhizobium sp., Azobacter sp., and Phyllobacterium sp. vectors.

In various embodiments, the genetic engineering context signature is a sequence from an expression vector including an origin of replication capable of replication in a bacterial cell. Exemplary bacterial origins of replications are F1, ColE1, Ori, OriC, pUC, Cori, pSC101, 15A, ARS, and OriT. Exemplary vectors include pBR322, the pUC series of vectors, the M13mp series of vectors, pACYC184, and the like.

In another embodiment, to identify a query sequence from a genetically engineered plant, animal, or human pathogen, for example, a genetic engineering organism can be identified.

The organism can be used for example for inserting, deleting, or knocking down genes, harboring and supporting synthetic genetic components through its modified molecular machinery, or for protein overexpression. In various embodiments, a genetic engineering organism can be a mammalian, insect, yeast, bacterial, or algal organism typically used in a protein expression system. Exemplary yeast organisms for expression include S. cerevisiae, Pichia pastoris, H. polymorpha, and Candida bodini. An exemplary insect expression system is the baculovirus system. A commonly used organism for expression in bacteria is E. coli.

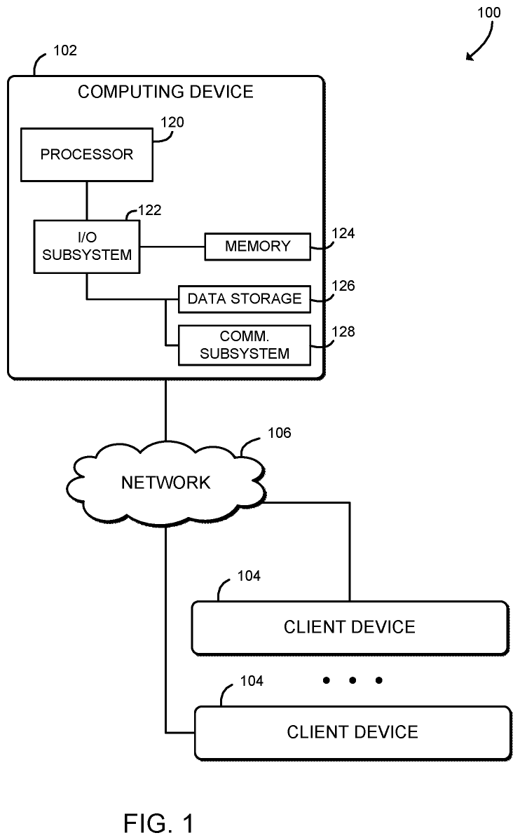

Referring now to FIG. 1, an illustrative system 100 includes a computing device 102 that may be in communication with one or more client devices 104 over a network 106. In use, as described further below, the computing device 102 receives one or more query sequences for a biological specimen (e.g., from a client device 104) and determines whether the query sequences are likely to indicate that the specimen is a result of genetic engineering. To perform this analysis, the computing device 102 may compare the query sequence to one or more predetermined databases of known genetic engineering proteins, genetic engineering organisms, or genetic engineering context signatures. Accordingly, the system 100 provides techniques to differentiate between engineered and non-engineered organisms, including pathogens, through nucleotide and amino acid sequence analysis, and further provides a strategy to identify genetic engineering context and functionality. Thus, the system 100 may improve identification of engineered organisms, and when applied to forensic bioinformatics may further assist in determining culpability, for example in relation to a deliberate engineered pathogen release.

Detection of artificial sequences contained within the chromosome or in extrachromosomal vectors may be accomplished through nucleic and amino acid sequencing and subsequent computational analyses to better elucidate distinct nucleic and amino acid sequence signatures associated with genetic engineering. Nucleic and amino acid sequence processing is generally considered intensive, especially when screening mixed microbial samples that may often be derived from patient specimen or other environmental matrices. However, high throughput sequencing tools and corresponding increases in computational power offered today afford more efficient processing of complex sequence data.

As described further below, information derived from nucleotide and amino acid sequence data may be used to identify taxa and potential functionality contained within biological samples. This information is especially critical within the context of identifying and understanding microbiological threats, as a rapid detection may ultimately lower the number of potential casualties in the event of biological warfare, and robust, high throughput methods for genetic engineering may increase the likelihood that engineered pathogens will be developed by terrorists or other adversaries.

The technology described herein relates to the utility of a software module which allows the user to identify indicators of genetic engineering in sequence datasets derived from a biological specimen. The module provides the user the capacity to flag key markers within sequences that are indicative of genetic modification. In addition to providing information with respect to taxonomic identification, this technology will help identify specific functions associated with the genetic engineering.

Referring again to FIG. 1, the computing device 102 may be embodied as any type of device capable of performing the functions described herein. For example, the computing device 102 may be embodied as, without limitation, a server, a rack-mounted server, a blade server, a workstation, a network appliance, a web appliance, a desktop computer, a laptop computer, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across the network 106 and operating in a public or private cloud. Accordingly, although the computing device 102 is illustrated in FIG. 1 as embodied as a single computing device, it should be appreciated that the computing device 102 may be embodied as multiple devices cooperating together to facilitate the functionality described below. As shown in FIG. 1, the illustrative computing device 102 includes a processor 120, an I/O subsystem 122, memory 124, a data storage device 126, and a communication subsystem 128. Of course, the computing device 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the computing device 102 may be embodied as any communication circuit, device, or collection thereof, capable of enabling communications between the computing device 102 and other remote devices. The communication subsystem 128 may be configured to use any one or more communication technology (e.g., wireless or wired communications) and associated protocols (e.g., Ethernet, InfiniBand® Bluetooth®, Wi-Fi®, WiMAX, 3G LTE, 5G, etc.) to effect such communication.

The client device 104 is configured to access the computing device 102 and otherwise perform the functions described herein. The client device 104 may be embodied as any type of computation or computer device capable of performing the functions described herein, including, without limitation, a computer, a laptop computer, a notebook computer, a tablet computer, a mobile computing device, a wearable computing device, a multiprocessor system, a server, a rack-mounted server, a blade server, a network appliance, a web appliance, a distributed computing system, a processor-based system, and/or a consumer electronic device. Thus, the client device 104 includes components and devices commonly found in a computer or similar computing device, such as a processor, an I/O subsystem, a memory, a data storage device, and/or communication circuitry. Those individual components of the client device 104 may be similar to the corresponding components of the computing device 102, the description of which is applicable to the corresponding components of the client device 104 and is not repeated herein so as not to obscure the present disclosure.

Each of the computing device 102 and/or the client devices 104 may be configured to transmit and receive data with each other and/or other devices of the system 100 over the network 106. The network 106 may be embodied as any number of various wired and/or wireless networks. For example, the network 106 may be embodied as, or otherwise include, a wired or wireless local area network (LAN), a wired or wireless wide area network (WAN), a cellular network, and/or a publicly-accessible, global network such as the Internet. As such, the network 106 may include any number of additional devices, such as additional computers, routers, stations, and switches, to facilitate communications among the devices of the system 100.

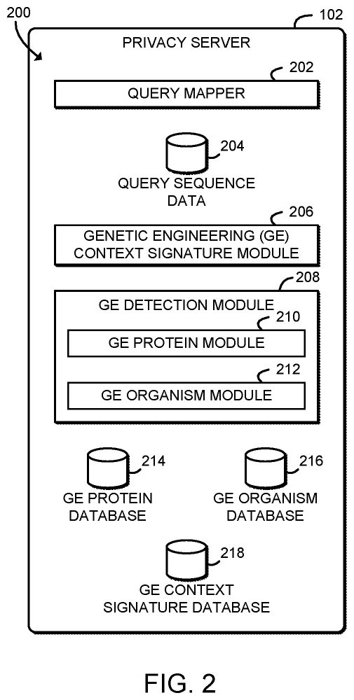

Referring now to FIG. 2, in the illustrative embodiment, the computing device 102 establishes an environment 200 during operation. The illustrative environment 200 includes query mapper 202, a genetic engineering (GE) context signature module 206, and a GE detection module 208. The various components of the environment 200 may be embodied as hardware, firmware, software, or a combination thereof. As such, in some embodiments, one or more of the components of the environment 200 may be embodied as circuitry or a collection of electrical devices (e.g., query mapper circuitry 202, GE context signature circuitry 206, and/or GE detection circuitry 208). It should be appreciated that, in such embodiments, one or more of those components may form a portion of the processor 120, the memory 124, the data storage 126, and/or other components of the computing device 102.

The query mapper 202 is configured to receive a query sequence for a biological specimen. The query sequence may be stored in or otherwise represented as query sequence data 204. The query sequence may comprise an amino acid sequence or a nucleotide sequence. In some embodiments, the query mapper 202 is further configured to determine an alignment of the query sequence against a predetermined database of sequences indicative of genetic engineering. In some embodiments, the query mapper 202 is further configured to determine an alignment of the query sequence for regions of interest. Each region of interest comprises a part of a whole protein translated region. The region of interest may comprise a protein that is indicative of genetic engineering, a predetermined protein sequence of interest, a protein associated with a biologically threatening function, and/or a predetermined protein.

The GE detection module 208 is configured to determine whether a similarity score associated with the alignment against the predetermined database of sequences indicative of genetic engineering has a predetermined relationship to a predetermined threshold, and to indicate presence of genetic engineering in response to determining that the similarity score has the predetermined relationship to the predetermined threshold. In some embodiments, the predetermined database may be a GE protein database 214, which comprises a database indicative of proteins, wherein each protein of the database 214 is indicative of genetic engineering. The proteins may include selectable markers, reporters, transcription regulators, post-translation regulators, gene editing/delivery proteins, plasmid replication proteins, protein couplers, protein folders, polymerases, and/or viral packaging/assembly proteins. Selectable markers may include a gene-encoded function that confers a selectable trait, wherein the trait may include a specific antibiotic resistance, a toxin, an antitoxin, and/or an auxotrophy marker. Reporters may include an enzymatic reporter, a direct optical reporter, and/or an analyte sensor. Transcription regulators may include a repressor and/or an activator. In some embodiments, the predetermined database may be GE organism database 216, which comprises a database indicative of organisms, wherein each organism of the database is indicative of genetic engineering. The organisms may include model organisms, delivery organisms, chassis/cloning organisms, and/or targeted protein overexpression organisms. In some embodiments, those functions of the GE detection module 208 may be performed by one or more sub-modules, such as a GE protein module 210 and/or a GE organism module 212.

The GE context signature module 206 is configured to determine whether a match for a genetic engineering context signature exists adjacent to a region of interest of the query sequence. The genetic engineering context signature comprises a sequence selected from a predetermined database of sequences indicative of genetic engineering context signatures. The GE context signature module 206 is further configured to indicate presence of the genetic engineering context signature in response to determining that the match exists. Determining whether the match for the genetic engineering context signature exists may include searching upstream or downstream of the region of interest in the query sequence, which may include searching upstream or downstream of the region of interest over a predetermined search range. The predetermined search range is associated with the genetic engineering context signature. The predetermined database of sequences indicative of genetic engineering context signatures may be GE context signature database 218. The genetic engineering context signatures may include upstream regulatory elements, downstream regulatory elements, and/or tags. Upstream regulatory elements may include a promoter, a ribosome binding site, an operator that contributes to transcript regulation, and/or an enhancer. Downstream regulatory elements may include a terminator, a polyA site, a woodchuck hepatitis virus posttranscriptional regulatory element (WPRE), a CTE, and/or an LTR. Tags may include a purification/epitope tag, a cleavage sequence, and/or a targeting sequence.

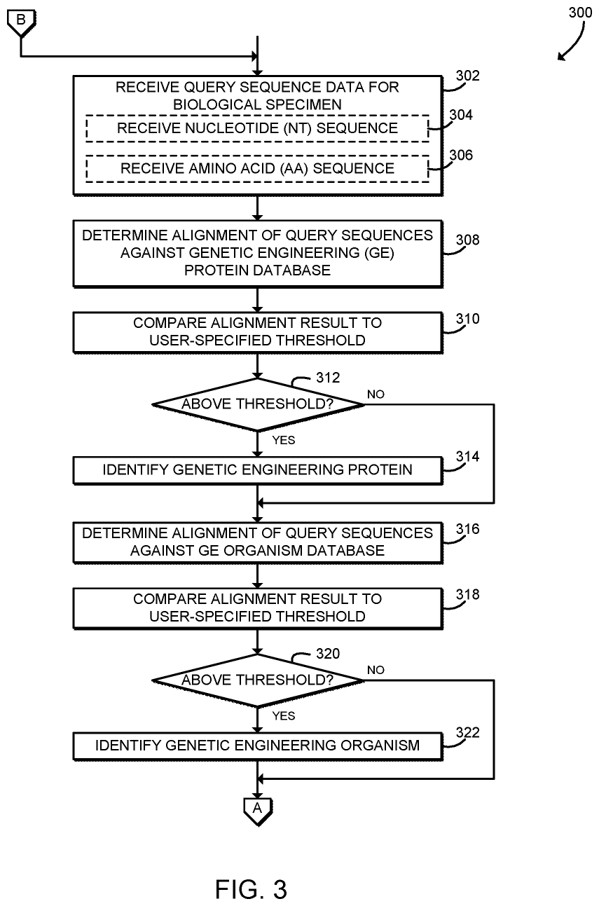

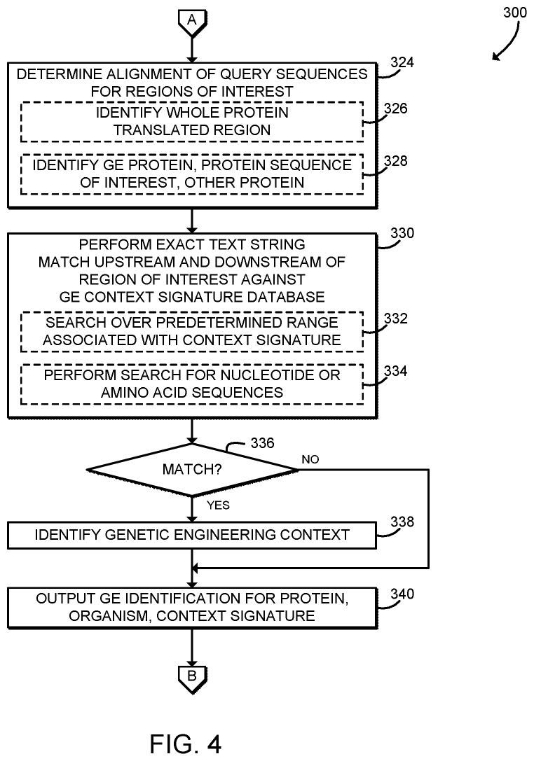

Referring now to FIGS. 3 and 4, in use, the computing device 102 may execute a method 300 for detecting genetic engineering proteins, organism, and context signatures. It should be appreciated that, in some embodiments, the operations of the method 300 may be performed by one or more components of the environment 200 of the computing device 102 as shown in FIG. 2. The method 300 begins with block 302, in which the computing device 102 receives query sequence data associated with a biological specimen. The query sequence data may include computer data describing a genetic sequence, proteomic sequence, gene, plasmid, or other genetic material. The query sequence data may be generated in a variety of scenarios, including, for example, trace detection of threats from a wipe sample, deep analysis of a single sequence, analysis of a digital data scrape, a metagenomics field sample (e.g., biosurveillance), comparison to lab-based analysis, or other sampling scenario. In an embodiment, the query sequence data may be received from one or more client devices 104, for example through submission to a web application or other server application executed by the computing device 102. Additionally, or alternatively, in some embodiments the computing device 102 may receive the query sequence data from a local or remote user through a user interface, from a sequencing machine, or from another sequence source. In some embodiments, in block 304 the computing device 102 may receive the query sequence data as a nucleotide sequence. In some embodiments, in block 306 the computing device 102 may receive the query sequence data as an amino acid sequence.

In block 308, the computing device 102 determines an alignment of one or more query sequences against the GE protein database 214. Determining the alignment identifies sequences within the GE protein database 214 that are similar to the query sequence. Additionally, when determining the alignment, the computing device 102 may determine a score, a similarly value, a confidence value, or another quantitative measure of similarity between the query sequence and one or more sequences within the GE protein database 214. The computing device 102 may use any local alignment, global alignment, or other genetic sequence alignment algorithm to align the query sequences against the GE protein database 214.

The GE protein database 214 includes data describing sequences of proteins that are known to be used in genetic engineering. Such proteins may include proteins used for selection, reporting, protein purification, or other genetic engineering purposes. Coding sequences for the proteins included in the GE protein database 214 may be described in published literature and/or known databases as being a component of a vector and/or other modular part during genetic engineering. For example, genetic engineering proteins may include selectable markers, reporters, transcription regulators, post-translation regulators, gene editing/delivery proteins, plasmid replication proteins, protein couplers, protein folders, polymerases, and/or viral packaging/assembly proteins.

Selectable markers may include a gene-encoded function that confers a selectable trait. As an example, a selectable marker may confer resistance to a specific antibiotic. As another example, a selectable marker may be composed of a toxin gene and its cognate antitoxin. As another example, a selectable marker may require a specific metabolite for growth or death (e.g., uracil auxotroph systems that enable selection through metabolic manipulation of the cell).

Reporters may include a gene-encoded function that is used in genetic engineering to indicate target gene transformation, expression of target gene, gene-gene interaction, or activity of a promoter or other genetic element. Reporter gene activities are easily measured through optical or other means. For example, an enzymatic reporter is a type of reporter in which the encoded gene is an enzyme such as beta galactosidase. The assay readout may be optically measured or measured by other means (e.g., radiological). As another example, a direct optical reporter is a type of reporter in which the encoded gene is a luminescent or other protein that can be directly measured optically. As yet another example, an analyte sensor is a type of reporter in which the encoded gene is a sensor of a specific analyte (e.g., calmodulin).

Transcription regulators may include a gene-encoded function that enables repression or activation of gene expression through binding of DNA elements upstream of the gene. Often, regulators are contained within operons. Transcription regulators may include repressors, which are a regulator protein that binds to the operator (a genetic sequence between the promoter and the expressed genes in an operon), thereby impeding RNA polymerase and thus gene expression. Repressors are often found in genetic engineering in combination with reporter genes or genes of interest to control gene expression. As another example, transcription regulators may include activators, which are a regulator protein that increases gene transcription, typically by binding to DNA elements upstream of a gene.

Post-translational regulators may include proteins that regulate the abundance of a target protein through promoting or avoiding degradation (e.g., ClpXP system or Ubiquitin).

Gene editing/delivery proteins may include a gene-encoded function that enables specific gene manipulation such as nucleases/recombinase or aiding in delivery of genetic material to different cell compartments (e.g., CRISPR, TALENS, Exonucleases, Cre recombinase, histone H2B). Often, such elements are encoded in vectors.

Plasmid replication proteins may include specific proteins involved in DNA replication origin or replication in plasmids. Such proteins can be encoded in broad host range plasmids (i.e., host-independent).

Protein couplers may include a specific function that leverages specific protein-protein or protein-ligand affinity interaction. Often, such elements are found in vectors coupled to proteins of interest to increase solubility or aid in purification, detection (e.g., streptavidin, maltose binding protein), display (e.g., coat protein for phage display), or are recombinantly produced for affinity resins (e.g., Protein A).

A protein folder may include a protein function used during protein expression to aid in folding the target protein correctly. A polymerase may include a specific polymerase such as T7 polymerase used in genetic engineering for protein production that may be found in vectors or other mobile genetic elements. Viral packaging/assembly proteins may include proteins used in the packaging of viruses to enable replication with a host for GE purposes such as creating stable cell lines (e.g., proteins that aid in packaging of human immunodeficiency virus in stable cell lines).

Still referring to FIG. 3, in block 310, the computing device 102 compares each alignment result to a user-specified threshold. As described above, the alignment results may include a score or other quantitative measure of similarity between the query sequence and one or more sequences within the GE protein database 214. The user-specified threshold may specify a minimum score above which the query sequence is considered to match a sequence in the GE protein database 214. Of course, in other embodiments, the user-specified threshold may have a different predetermined relationship to the alignment results, for example a maximum score when lower scores indicate greater similarity. The user-specified threshold may be provided by the user when submitting the query sequence or may be configured ahead of time, for example in predetermined configuration settings. In block 312, the computing device 102 determines whether an alignment result is above the threshold or otherwise has the predetermined relationship to the threshold. If not, the method 300 skips ahead to block 316. If the alignment result is above the threshold, the method 300 advances to block 314.

In block 314, the computing device 102 identifies a genetic engineering protein in the query sequence. The computing device 102 may, for example, set a flag or otherwise indicate the presence of genetic engineering. As another example, the computing device 102 may record or otherwise indicate the particular genetic engineering protein from the GE protein database 214 that was identified in the query signature. As described further below, the indication of genetic engineering protein may be combined with one or more other indications of genetic engineering that may be present in the query sequence. After identifying the genetic engineering protein, the method 300 advances to block 316.

In block 316, the computing device 102 determines an alignment of one or more query sequences against the GE organism database 216. Determining the alignment identifies sequences within the GE organism database 216 that are similar to the query sequence. Additionally, as described above, when determining the alignment, the computing device 102 may determine a score, a similarly value, a confidence value, or another quantitative measure of similarity between the query sequence and one or more sequences within the GE organism database 216. The computing device 102 may use any local alignment, global alignment, or other genetic sequence alignment algorithm to align the query sequences against the GE organism database 216.

The GE organism database 216 includes data describing sequences associated with organisms that are known to be used in genetic engineering. Genetic engineering organisms may include those used as model organisms, delivery vehicles, cloning, and/or protein production. A model organism may be an extensively studied organism that has a short regeneration period, a fully characterized genome, and contains attributes similar to humans that can be used for studying a specific traits, diseases, or phenotypes. A delivery organism may be an organism used for inserting, deleting, or knocking down genes for gene therapy or genome editing. A chassis or cloning organism may be an organisms or cell type capable of harboring and supporting synthetic genetic components through its natural or modified molecular machinery, such as transcriptional and translational systems. A protein over-production organism/heterologous expression organism may be an organism or cell type (e.g., bacteria, yeast, insect, or mammalian cells) which is transformed with vectors for targeted protein overexpression.

In block 318, the computing device 102 compares each alignment result to a user-specified threshold. As described above, the alignment results may include a score or other quantitative measure of similarity between the query sequence and one or more sequences within the GE organism database 216. The user-specified threshold may specify a minimum score above which the query sequence is considered to match a sequence in the GE organism database 216. Of course, in other embodiments, the user-specified threshold may have a different predetermined relationship to the alignment results, for example a maximum score when lower scores indicate greater similarity. The user-specified threshold may be provided by the user when submitting the query sequence or may be configured ahead of time, for example in predetermined configuration settings. In block 320, the computing device 102 determines whether an alignment result is above the threshold or otherwise has the predetermined relationship to the threshold. If not, the method 300 skips ahead to block 324, shown in FIG. 4. If the alignment result is above the threshold, the method 300 advances to block 322.

In block 322, the computing device 102 identifies a genetic engineering organism in the query sequence. The computing device 102 may, for example, set a flag or otherwise indicate the presence of genetic engineering. As another example, the computing device 102 may record or otherwise indicate the particular genetic engineering organism from the GE organism database 216 that was identified in the query signature. As described further below, the indication of genetic engineering organism may be combined with one or more other indications of genetic engineering that may be present in the query sequence. After identifying the genetic engineering organism, the method 300 advances to block 324, shown in FIG. 4.

Referring now to FIG. 4, in block 324 the computing device 102 determines an alignment of one or more query sequences for regions of interest. The computing device 102 may, for example, identify the start and stop for each region of interest within the query sequence. In some embodiments, in block 326 the computing device 102 identifies a whole protein translated region (TR) within the query sequence. Each region of interest may include part or all of the translated region. In some embodiments, in block 328 the computing device 102 may identify a GE protein, a protein sequence of interest, or another protein for each region of interest. For example, the computing device 102 may identify GE proteins based on the GE protein database 214. As another example, the computing device 102 may identify one or more predetermined protein sequences of interest, such as sequences that are associated with biologically threatening functions.

In block 330, the computing device 102 performs a search upstream or downstream of the region of interest against signatures in the GE context signature database 218. The computing device 102 may search for a matching signature in the GE context signature database 218, for example a promoter with high sequence identity, or an exact text string match. The GE context signature database 218 includes context signatures, which are relatively small, predetermined sequences that are known to be used in genetic engineering, for example as a component of a vector and/or another modular part used during genetic engineering. Context signatures may include sequences found either upstream or downstream of one or more coding sequences. The context signatures may regulate transcription of the gene and/or aid cellular localization or purification of the protein product. These sequences may be described in published literature and/or databases as being used in genetic engineering.

Genetic engineering context signatures may include upstream regulatory elements, downstream regulatory elements, and/or tags. Upstream regulatory elements may include DNA sequences found upstream of a coding gene that regulate transcription and/or protein expression. For example, such upstream regulatory elements may include a promoter, which is a DNA sequence that initiates transcription of a gene downstream via binding of RNA polymerase and/or transcription factors (e.g., a T7 promoter). As another example, upstream regulatory elements may include a ribosome binding site (RBS), that is, those RBSs that are not found ubiquitously in nature. As still further examples, upstream regulatory elements may include other DNA regulator elements, such as operators, that contribute to transcription regulation (e.g., a “protein_bind” feature in Addgene such as the lac operator, which binds to lac repressor), a TRE response element which is a binding site for activator protein, “LTR” features, 5′UTRs, and/or insulators. Upstream regulatory elements may include enhancers, which are DNA sequences typically found upstream of a promoter that binds transcription factors to increase transcription. Enhancers are more common in eukaryotic systems than prokaryotic systems.

Downstream regulatory elements may include DNA sequences found downstream of a coding gene that regulate transcription and/or protein expression. For example, such downstream regulatory elements may include a terminator, which is a DNA sequence downstream of a coding sequence that triggers processes in the transcribed RNA to terminate transcription. As another example, downstream regulatory elements may include a polyA site/polyA signal, which is a DNA sequence that encodes for a poly(A) stretch, which may be important for nuclear export, translation, and stability of mRNA. The poly A site typically occurs immediately before the terminator. While more common in eukaryotes, polyadenylation may also occur in prokaryotes. As still further examples, further downstream regulatory elements may include woodchuck hepatitis virus posttranscriptional regulatory element (WPRE), which enhances expression, 3′UTRs, insulators, CTE, and/or LTRs.

Tags may include an amino acid (AA) sequence (coding sequence) found at the N-terminal or C-terminal end of an engineered protein to target the protein to specific cellular locations and/or aid in protein purification. For example, tags may include a purification/ epitope tag, which is an amino acid tag that enables purification or detection using specific resins, antibodies, and/or proteins (e.g., His×6 tag, HA tag, or other tags). As another example, tags may include a cleavage sequence, which is a specific sequence that can be cleaved to release the target protein(s) of interest from other components (e.g., TEV protease or a self-cleaving peptide). As another example, tags may include a targeting sequence, which is a specific sequence that targets the protein to a specific cellular location (e.g., nuclear localization sequence).

Still referring to FIG. 4, in some embodiments, in block 332, the computing device 102 searches over a predetermined range associated with each context signature. The range may be specified as a search start, search end, and/or a search length, and may be specified relative to the start of the region of interest for upstream searches, or relative to the end of the region of interest for downstream searches. The search range may be based on the particular type of context signature, and may be selected such that a relatively large proportion of known context signatures (e.g., from the literature) will be found within the search range. For example, identified literature sources suggest promoters and enhancers are typically a few hundred base pairs in length, with promoters usually located immediately upstream of the transcription start site (typically within 50 bps). Downstream terminators are typically within 100 bps of the stop codon and may overlap with the gene. Examples of predetermined search ranges for various context signature types are shown below in Table 1. In some embodiments, in block 334, the computing device 102 performs the search for context signatures that are nucleotide sequences or amino acid sequences.

TABLE 1
Search ranges for genetic engineering context signatures.

| Feature | | Direction | Range | Range | |
|---|---|---|---|---|---|
| Type | Example | from CDS | Start | End | Rationale |
| Promoter | T7 promoter | Upstream | −600 | 0 | Majority of Addgene |
| | | | | | features in this |
| | | | | | range; distribution |
| | | | | | suggests ~600 is a good |
| | | | | | break point |
| Enhancer | CMV enhancer | Upstream | −800 | −400 | Found upstream of |
| | | | | | promoters; estimated >90% |
| | | | | | of analyzed Addgene |
| | | | | | features in this range |
| Protein | Lac operator | Upstream | −500 | 0 | Found downstream of |
| Bind | | | | | promoters; estimated >90% |
| | | | | | of analyzed Addgene |
| | | | | | features in this range |
| Misc. | Internal ribosome | Upstream | −600 | 0 | Variable; may be set to |
| | entry site (IRES) | | | | max upstream distance |
| RBS/ | Shine-Dalgarno | Upstream | −50 | 10 | Found immediately |
| Regulatory | sequence | | | | upstream of start |
| | | | | | codon; may overlap |
| | | | | | with start codon |
| 5′UTR | 5′UTR of | Upstream | −250 | 0 | Estimated based on |
| | thymidine kinase | | | | examples identified in |
| | | | | | Addgene |
| Misc. | WPRE | Downstream | 500 | 750 | Estimated >90% |
| | | | | | analyzed Addgene |
| | | | | | features in this range |
| LTR | Retroviral long | Downstream | 800 | 1,200 | Estimated >90% |
| | terminal repeats | | | | analyzed Addgene |
| | (LTRs) | | | | features in this range |
| PolyA | bGH poly(A) | Downstream | 0 | 500 | Estimated >90% |
| Signal/Site | signal | | | | analyzed Addgene |
| | | | | | “site” features in this |
| | | | | | range; poly A site |
| | | | | | found immediately |
| | | | | | downstream of poly A |
| | | | | | signal (high overlap in |
| | | | | | range) |
| Terminator | T7 terminator | Downstream | −25 | 500 | Estimated >90% |
| | | | | | analyzed Addgene |
| | | | | | features in this range |
| 3′UTR | cspA 3′UTR | Downstream | 0 | 500 | Examples identified |
| | | | | | occur <400 bps after |
| | | | | | stop codon; set to |
| | | | | | conservative max of |
| | | | | | 500 |
| Tag | His6 tag | Either | −60 | 60 | Tags may be added |
| | | | | | immediately before or |
| | | | | | after start/stop codon; |
| | | | | | set to ~max length of |
| | | | | | tags |

In block 336, the computing device 102 determines whether a match for a context signature was found. If not, the method 300 skips ahead to block 340, described below. If so, the method 300 advances to block 338.

In block 338, the computing device 102 identifies a genetic engineering context signature in the query sequence. The computing device 102 may, for example, set a flag or otherwise indicate the presence of genetic engineering. As another example, the computing device 102 may record or otherwise indicate the particular genetic engineering context signature from the GE context signature database 218 that was identified in the query signature. This context signature may be associated with a particular function or may otherwise provide insight into the genetic engineering that was performed. As described further below, the indication of genetic engineering context signature may be combined with one or more other indications of genetic engineering that may be present in the query sequence. After identifying the genetic engineering context signature, the method 300 advances to block 340, shown in FIG. 4.

In block 340, the computing device 102 outputs any genetic engineering identification data associated with GE proteins, GE organisms, or GE context signatures determined as described above. The computing device 102 may, for example, provide a web page or other report to a client device 104 or otherwise provide the identification data to a user. As another example, the computing device 102 may provide the genetic engineering identification data to one or more additional genetic sequence analysis modules executed by the computing device 102. After outputting the genetic engineering identification data, the method 300 loops back to block 302, shown in FIG. 3, in order to process additional query signatures.

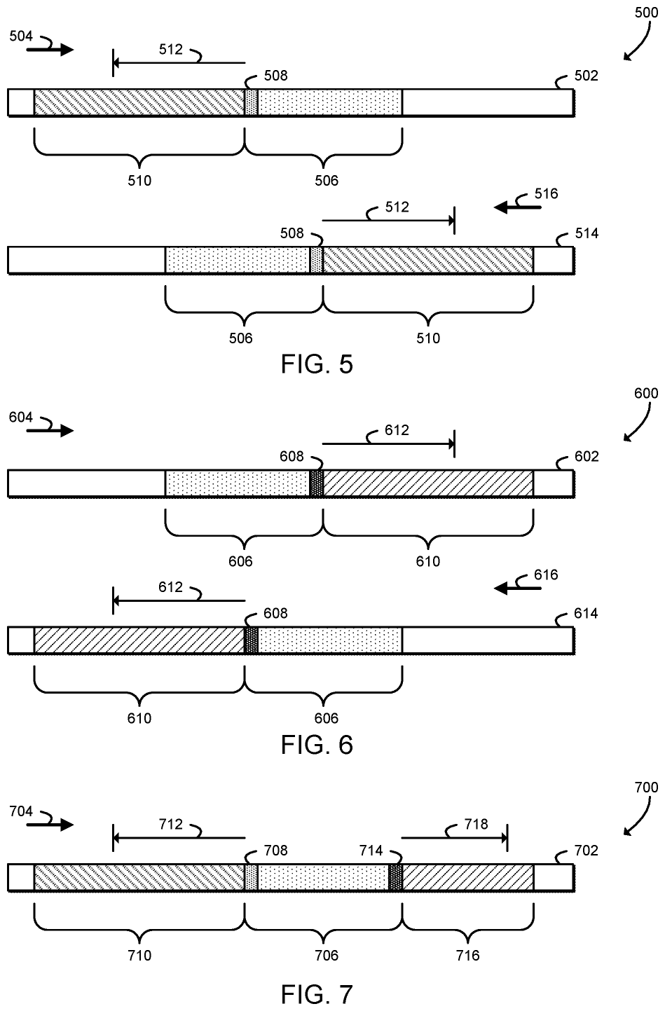

Referring now to FIG. 5, diagram 500 illustrates one potential embodiment of a search for genetic engineering context signatures upstream of the region of interest. The diagram 500 shows a query sequence 502, which is illustratively a nucleotide sequence. The query sequence 502 is processed in the forward frame, as illustrated by arrow 504. A region of interest 506 is identified in the query sequence 502. A start 508 of the region 506 is identified. Illustratively, the start 508 is the first base pair of the region 506, and may be assigned an index of zero. The computing device 102 may search an upstream range 510 relative to the region 506. More particularly, the computing device 102 may search the upstream range 510 within a search range 512 of the start 508 of the range 506. The search range 512 is illustratively a predetermined length associated with each type of context signature. For example, given a search range 512 of 50 base pairs, the upstream search range may be expressed as [−50, 0]. Continuing that example, in some embodiments, the context signature may not overlap the region of interest 506, so the search range may be reduced by the length of the context signature. In those embodiments, the illustrative range may be expressed as [−50, 0−length(signature)].

The diagram 500 also shows a nucleotide query sequence 514, which is processed in the reverse frame as illustrated by arrow 516. The query sequence 514 similarly includes a region of interest 506 with a start 508 and an upstream region 510 with associated search range 512. When searching for signatures in the upstream region 510 in the reverse frame 516, the signatures may be reverse complemented.

Referring now to FIG. 6, diagram 600 illustrates another potential embodiment of a search for genetic engineering context signatures downstream of the region of interest. The diagram 600 shows a query sequence 602, which is illustratively a nucleotide sequence. The query sequence 602 is processed in the forward frame, as illustrated by arrow 604. A region of interest 606 is identified in the query sequence 602. A stop 608 of the region 606 is identified. Illustratively, the stop 608 is the first base pair of the stop codon for the region 606, and may be assigned an index of zero. The computing device 102 may search a downstream range 610 relative to the region 606. More particularly, the computing device 102 may search the downstream range 610 within a search range 612 of the stop 608 of the range 606. The search range 612 is illustratively a predetermined length associated with each type of context signature. For example, given a search range 612 of 50 base pairs, the downstream search range may be expressed as [3, 50]. Continuing that example, in some embodiments, the search range may be reduced by the length of the context signature. In those embodiments, the illustrative range may be expressed as [3, 50−length(signature)].

The diagram 600 also shows a nucleotide query sequence 614, which is processed in the reverse frame as illustrated by arrow 616. The query sequence 614 similarly includes a region of interest 606 with a stop 608 and a downstream region 610 with associated search range 612. When searching for signatures in the downstream region 610 in the reverse frame 616, the signatures may be reverse complemented.

Referring now to FIG. 7, diagram 700 illustrates one potential embodiment of a search for genetic engineering context signatures upstream or downstream of the region of interest. The diagram 700 shows a query sequence 702, which is illustratively an amino acid sequence. The query sequence 702 is processed in the forward frame, as illustrated by arrow 704. A region of interest 706 is identified in the query sequence 702. A start 708 of the region 706 is identified. Illustratively, the start 708 is the first amino acid of the region 706, and may be assigned an index of zero. The computing device 102 may search an upstream range 710 relative to the region 706. More particularly, the computing device 102 may search the upstream range 710 within a search range 712 of the start 708 of the range 706. The search range 712 is illustratively a predetermined length associated with each type of context signature. For example, given a search range 712 of 66 amino acids, an upstream search range may be expressed as [−33, 33]. Continuing that example, the search range may be reduced by the length of the context signature. In those embodiments, the illustrative range may be expressed as [−33, 33−length(signature)].

As shown in FIG. 7, downstream searches of the query sequence 702 may also be performed. As shown, a stop 714 of the region 706 is identified. Illustratively, the stop 708 is the last amino acid for the region 706, and may be assigned an index of zero. The computing device 102 may search a downstream range 716 relative to the region 706. More particularly, the computing device 102 may search the downstream range 716 within a search range 718 of the stop 714 of the range 706. The search range 718 is illustratively a predetermined length associated with each type of context signature. Additionally, as shown in FIG. 7, the illustrative query sequence 702 is processed in the forward frame 704. Appropriate adjustments for sequences in the reverse frame may be made, similar to the searches described above in connection with FIGS. 5 and 6.

1. A method for identifying genetic engineering, the method comprising:
receiving, by a computing device, a query sequence for a biological specimen;
determining, by the computing device, an alignment of the query sequence for regions of interest, wherein each region of interest comprises a part of a whole protein translated region;
determining, by the computing device, whether a match for a genetic engineering context signature exists adjacent to a region of interest of the query sequence, wherein the genetic engineering context signature comprises a sequence selected from a predetermined database of sequences indicative of genetic engineering context signatures; and
indicating, by the computing device, presence of the genetic engineering context signature in response to determining that the match exists.

2. The method of claim 1, wherein determining whether the match for the genetic engineering context signature exists adjacent to the region of interest comprises searching upstream or downstream of the region of interest in the query sequence.

3. The method of claim 2, wherein searching upstream or downstream of the region of interest comprises searching over a predetermined search range, wherein the predetermined search range is associated with the genetic engineering context signature.

4. The method of claim 1, wherein the region of interest comprises a protein associated with a biologically threatening function.

5. The method of claim 1, wherein the genetic engineering context signature comprises an upstream regulatory element, a downstream regulatory element, or a tag.

6. The method of claim 5, wherein the genetic engineering context signature comprises an upstream regulatory element, wherein the upstream regulatory element comprises a promoter, a ribosome binding site, an operator that contributes to transcript regulation, or an enhancer.

7. The method of claim 5, wherein the genetic engineering context signature comprises a downstream regulatory element, wherein the downstream regulatory element comprises a terminator, a polyA site, a woodchuck hepatitis virus posttranscriptional regulatory element (WPRE), a CTE, or an LTR.

8. The method of claim 5, wherein the genetic engineering context signature comprises a tag, wherein the tag comprises a purification/epitope tag, a cleavage sequence, or a targeting sequence.

9. A computing device for identifying genetic engineering, the computing device comprising:
a query mapper to (i) receive a query sequence for a biological specimen and (ii) determine an alignment of the query sequence for regions of interest, wherein each region of interest comprises a part of a whole protein translated region; and
a genetic engineering context module to (i) determine whether a match for a genetic engineering context signature exists adjacent to a region of interest of the query sequence, wherein the genetic engineering context signature comprises a sequence selected from a predetermined database of sequences indicative of genetic engineering context signatures, and (ii) indicate presence of the genetic engineering context signature in response to a determination that the match exists.

10. The computing device of claim 9, wherein to determine whether the match for the genetic engineering context signature exists adjacent to the region of interest comprises to search upstream or downstream of the region of interest in the query sequence.

11. The computing device of claim 10, wherein to search upstream or downstream of the region of interest comprises to search over a predetermined search range, wherein the predetermined search range is associated with the genetic engineering context signature.

12. The computing device of claim 9, wherein the genetic engineering context signature comprises an upstream regulatory element, a downstream regulatory element, or a tag.

13. A method for identifying genetic engineering, the method comprising:
receiving, by a computing device, a query sequence for a biological specimen;
determining, by the computing device, an alignment of the query sequence against a predetermined database of sequences indicative of genetic engineering;
determining, by the computing device, whether a similarity score associated with the alignment has a predetermined relationship to a predetermined threshold; and
indicating, by the computing device, presence of genetic engineering in response to determining that the similarity score has the predetermined relationship to the predetermined threshold.

14. The method of claim 13, wherein the predetermined database of sequences indicative of genetic engineering comprises a database indicative of proteins, wherein each protein of the database is indicative of genetic engineering.

15. The method of claim 14, wherein each protein comprises a selectable marker, a reporter, a transcription regulator, a post-translation regulator, a gene editing/delivery protein, a plasmid replication protein, a protein coupler, a protein folder, a polymerase, or a viral packaging/assembly protein.

16. The method of claim 15, wherein the protein comprises a selectable marker, and wherein the selectable marker comprises a gene-encoded function that confers a selectable trait, wherein the trait comprises a specific antibiotic resistance, a toxin, an antitoxin, or an auxotrophy marker.

17. The method of claim 15, wherein the protein comprises a reporter, and wherein the reporter comprises an enzymatic reporter, a direct optical reporter, or an analyte sensor.

18. The method of claim 15, wherein the protein comprises a transcription regulator, wherein the transcription regulator comprises a repressor or an activator.

19. The method of claim 13, wherein the predetermined database of sequences indicative of genetic engineering comprises a database indicative of organisms, wherein each organism of the database is indicative of genetic engineering.

20. The method of claim 19, wherein the organism comprises a model organism, a delivery organism, a chassis/cloning organism, or a targeted protein overexpression organism.
